(19) United States
(12) Patent Application Publication
(10) Pub. No.: US 2013/0309185 A1
(43) Pub. Date: Nov. 21, 2013

(54) COMPOSITIONS COMPRISING STABILIZED KETO- ENOL UV ABSORBERS

(71) Applicants: Hani M. Fares, Somerset, NJ (US); Donald I. Prettypaul, Englewood, NJ (US)

(72) Inventors: Hani M. Fares, Somerset, NJ (US); Donald I. Prettypaul, Englewood, NJ (US)

(21) Appl. No.: 13/991,795

(22) PCT Filed: Dec. 9, 2011
(86) PCT No.: PCT/US11/64110

§ 371 Date: Aug. 2, 2013

Related U.S. Application Data
(60) Provisional application No. 61/421,663, filed on Dec. 10, 2010.

(51) Int. Cl. A61K 8/35; A61Q 17/00

(52) U.S. Cl. CPC A61K 8/35; A61Q 17/00
USPC 424/60; 424/59; 424/70.9

(57) ABSTRACT

The present application provides compositions having a keto-enol UV absorber, such as avobenzone, stabilized from forming iron complexes. The stabilization is provided by a chelating polymer derived from at least one monomer having lactam or carboxylic acid functionality. In one embodiment, the compositions are sunscreens that resist staining fabrics when washed in hard water. In another embodiment, the compositions also comprise one or more iron-containing adjuvants, such as colorants, dyes, and pigments. By stabilizing the UV absorber, cosmetics can be formulated that impart broad-spectrum UV-A protection to the user. Additional ingredients can be added to the formulation, like other UV absorbers (for even broader UV-A and UV-B protection), moisturizers, actives, conditioning agents, and rheology modifiers.

### BACKGROUND

### Field

Avobenzone, also known as 1-(4-methoxyphenyl)-3-(4-tert-butylphenyl)propane-1,3-dione, is an absorber of ultraviolet (UV) radiation. It finds wide use in sunscreens, sunblocks, and other related products due to its ability to absorb the full spectrum of UV-A radiation, with an absorption maximum of 357 nm. Avobenzone was approved for cosmetic use in Europe in 1978, in the United States in 1988, and currently is used worldwide. It is available under a variety of trade names, including Escalol® 517, Parsol® 1789 (DSM Nutritional Products), and Eusolex® 9020 (Merck Group).

Despite its broad UV spectrum absorptivity, formulating with avobenzone can be challenging due in part to its chemical structure. Avobenzone is subject to keto-enol isomerization:

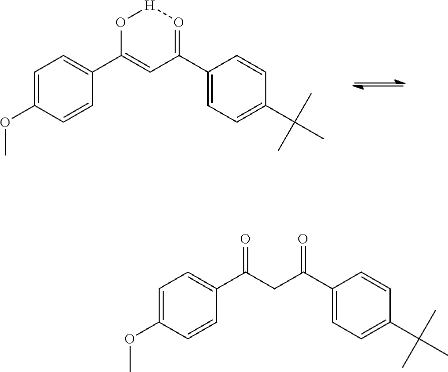

being facilitated by a variety of causes (e.g., solvent, other UV absorbers). Due to the weakly acidic nature of the enol form and electron resonance, an enolate anion forms:

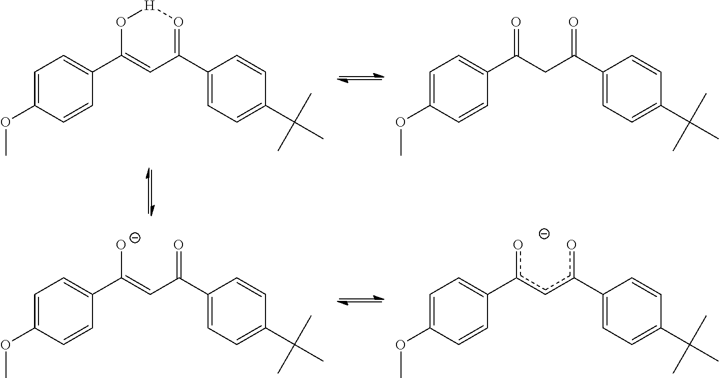

that can chelate with cations, like Fe²⁺, Fe³⁺, and others, producing a red-colored, water-insoluble complex. Additionally, the complex can chemically destabilize the avobenzone molecule so that it is subject to cleavage mechanisms. This isomerization is not unique to avobenzone, but is attributed to the diketone moiety that participates in the resonance:

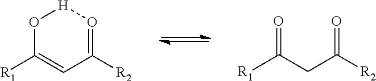

wherein R₁ and R₂ represent any group and can be the same or different.

Since cosmetics frequently utilize iron-containing compounds as colorants, astringents, and skin conditioning agents, it has been impossible for aesthetic and stability reasons to create UV-protecting cosmetics containing avobenzone. In fact, the sales brochure titled, “Parsol® 1789” by DSM Nutritional Products, which is hereby incorporated in its entirety by reference, specifically names iron and heavy metals as materials to be avoided.

Similarly, the use of avobenzone in sunscreens (including anhydrous spray products) is also fraught with this chelation problem. When fabrics having avobenzone-containing sunscreens are laundered in hard water (containing free iron), the fabric can be stained due to the avobenzone-iron complex. Many commercial sunscreens for UV-A protection advise staining may occur if fabrics containing the sunscreen (e.g., during application or transfer by wiping) are washed in water containing iron.

Hence, while there is a great need to formulate broad UV-A spectrum absorbance into cosmetics and sunscreens using avobenzone, it must be done so without encountering the above noted problems.

In addition to these chelation problems, many UV absorbers, including avobenzone, exhibit photolability, in which absorbed energy causes photodegradation and/or photoreactivity, and thus reduces its efficacy. Such photolability may result from irreversible is omerisms (i.e., keto-enol tautomerism and cis-trans isomerism), photocleavage, and/or photoaddition, and may be formulation sensitive, (e.g., blends of avobenzone and octinoxate). In particular, avobenzone is subject to bimolecular reactions (viz, via cleavage mechanisms) that alter the molecule's structure and decrease its energy absorption and, therefore, its effectiveness as an UV absorber. Other labile UV absorbers are known, and include p-aminobenzoic acid (PABA) derivatives, cinnamates, and dibenzoyl methane derivatives, all of which degrades over time, and reduce UV protection.

Hence, there exists a need to stabilize avobenzone and other UV absorbers from photodegradative effects.

One attempt to limit avobenzone complexes with iron has been the use of chelating agents, like ethylenediaminetetraacetic acid (EDTA), or one of its salts. Unfortunately, EDTA is very poorly soluble in water (around 0.05 mg/mL), and only reaches substantial solubility at pH greater than 8. However, EDTA buffers solutions, so that additional base is required to further increase its concentration. Efficient use of EDTA requires basic conditions that frequently cannot be met in cosmetics nor sunscreen formulations.

Other methods for stabilizing UV absorbers, and in particular UV absorbers, are known in the prior art, but have not solved the limitations facing avobenzone. For example, U.S. Pat. No. 4,868,246 discloses polymer chemistries having UV absorbers bonded to recurring units:

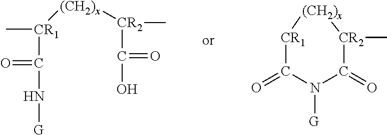

in the polymer backbone, on grafted side chains, as pendant units, or as combinations thereof. The group N-G is the residue of a primary amino or hydrazido substituted stabilizer group selected from (a) 2-hydroxybenzophenones, (b) 2-(2-hydroxyphenyl)-2H-benzotriazoles, (c) aryl salicylates, or (d) oxalic acid amides. A number of potential maleic anhydride polymers are potential stabilizers for the UV absorbers, and include: (a) styrene-maleic anhydride, (b) alternating copolymers of maleic anhydride and alpha-olefins, (c) alkyl vinyl ethers and maleic anhydride, (d) maleic anhydride modified polyolefins, (e) maleic anhydride adducts of hydrogenated polymers or copolymers, and (f) maleic anhydride adducts of EPDM.

A similar approach is disclosed in U.S. Pat. No. 4,857,596 for thermally stabilizing antioxidants.

Two radiation-absorbing polymer chemistries are disclosed in U.S. Pat. No. 6,255,405. The polymeric compositions comprise two recurring units, the first having the formula:

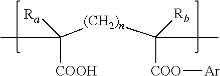

wherein Rₐ and R_{b} may be the same or different and represent hydrogen, an alkyl group or other organic groups, Ar represents an organic UV absorber, and n represents 0 or an integer of 1 or more; and the second recurring unit having the formula:

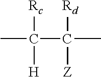

wherein R_{c} and R_{d} may be the same or different and each represents hydrogen, an alkyl group, a carboxyl group, or other organic groups, and Z represents hydrogen, a substituted or non-substituted alkoxyl group, a substituted or non-substituted alkyl group, a halogen atom, —CN, an alkylcarbonyloxy group, an imide group, a substituted or non-substituted carbamoyl group, a substituted or non-substituted oxycarbonyl group, or a substituted or non-substituted phenyl group.

Also known is the sunscreen formula of U.S. Patent Application Publication No. WO 2009/036030, which has a tetrapolymer of methacrylic acid, methyl methacrylate, butyl acrylate and cetyl eicosinyl methacrylate and (b) an acrylic acid/vinyl pyrrolidone crosspolymer. Avobenzone is one example of a UV absorber suitable for use in the compositions disclosed in the '030 publication.

Color cosmetic compositions are the subject of U.S. Pat. No. 7,799,321. This patent discloses compositions having a rheology modifier and film-former copolymer that is compatible with iron oxides. However, the disclosed compositions do not contain any UV absorber, and the stabilization of keto-enol UV absorbers is not provided.

Despite advances in designing UV absorbers and in developing formulation blends, there remains a consumer demand to create stable cosmetics that provide UV-A protection from avobenzone, and also to offer sunscreens with avobenzone that do not stain clothes when washed in hard water. There also remains a need for UV absorbers having enhanced performance, especially for single compositions that provide full UV-spectrum protection, enhanced water-resistance, reduced tendency for skin penetration, and improved stability especially for labile UV absorbers. The compositions disclosed herein uniquely accomplish these properties.

### SUMMARY

Compositions have been newly discovered that prevent keto-enol UV absorbers from forming a complex with iron. These compositions formulate a suitable UV absorber, such as avobenzone, with a chelating polymer. Six different chelating polymers are described, monomer units responsible for the activity are identified. Since little or no avobenzone-iron complex is formed, pigmented cosmetics can be created that provide broad UV-A protection.

The present application also provides sunscreens having these keto-enol UV absorbers that do not stain fabrics when washed in hard water having iron. Other ingredients can fill-out the compositions disclosed herein, such as moisturizers, emollients, fragrances, and other UV absorbers in order to provide full UV-A and UV-B protection.

### DETAILED DESCRIPTION

Before describing particular embodiments of the invention, it is helpful to define a few terms first as they are used in this description.

The term “monomer” refers to a repeating structural unit of a polymer. A monomer is a low molecular weight compound that can form covalent chemical bonds with itself and/or with other monomers, resulting in a polymer.

The term “polymer” refers to a compound comprising repeating structural units (monomers) connected by covalent chemical bonds. The definition includes oligomers. Polymers may be further derivatized (example by hydrolysis), crosslinked, grafted or end-capped. Non-limiting examples of polymers include copolymers, terpolymers, quaternary polymers, and homologues. A polymer may be a random, block, or an alternating polymer, or a polymer with a mixed random, block, and/or alternating structure.

The term “copolymer” refers to a polymer consisting essentially of two types of repeating structural units (monomers).

The term “terpolymer” refers to a polymer consisting essentially of three types of repeating structural units (monomers).

The term “(meth)acrylate” refers to both acrylate and methacrylate. Similarly, the term “(meth)acrylamide” refers to both acrylamide and methacrylamide.

The terms “ultraviolet” and “UV” refer to electromagnetic radiation, especially solar electromagnetic radiation, with a wavelength from about 100 nm to about 400 nm, and includes the UV-A, UV-B, and UV-C subclassifications of such radiation.

The term “UV-A” refers to ultraviolet electromagnetic radiation with a wavelength from about 320 nm to about 400 nm, and includes UV-A1 (from about 340 nm to about 400 nm) and UV-A2 (from about 320 nm to about 340 nm).

The term “UV-B” refers to ultraviolet electromagnetic radiation with a wavelength from about 290 nm to about 320 nm.

The term “UV-C” refers to ultraviolet electromagnetic radiation with a wavelength from about 200 nm to about 290 nm.

The term “UV absorber” refers to compound that absorb, reflect, and/or scatter UV radiation.

The term “keto-enol UV absorber” refers to UV absorbers having at least one diketone moiety. A non-limiting example of a preferred keto-enol UV absorber is avobenzone.

Personal care compositions refer to such illustrative non-limiting compositions as skin, sun, oil, hair, cosmetic, and preservative compositions, including those to alter the color and appearance of the skin. Other personal care compositions are those that enhance flexibility in styling, durable styling, increased humidity resistance for hair, skin, and color cosmetics, sun care water-proof/resistance, wear-resistance, and thermal protecting/enhancing compositions.

The term “sunscreen” refers to personal care and/or pharmaceutical formulations comprising an effective amount of one or more UV-absorbing compounds. Sunscreen formulations include beach and non-beach products that are applied to the face, décolleté, lips, and skin to treat and/or protect against erythema, burns, wrinkles, lentigo (“liver spots”), skin cancers, keratotic lesions, and cellular changes of the skin; and to hair to treat and/or protect against color changes, lack of luster, tangles, split ends, unmanageability, and embrittlement. Other categories of sunscreens that fit into this definition are sun blocks, all-day protection formulas, baby sun care, and tanning prepations that have UV absorber(s).

The term “performance chemicals application” refers to any application that is not a personal care or pharmaceutical application. Examples of performance chemicals applications include, but are not limited to: adhesive, agricultural, cleaning, coating, construction material, encapsulation, ink, membrane, personal care, printing, plastic, or packaging composition.

All percentages, ratio, and proportions used herein are based on a weight basis unless other specified.

### Description of the Compositions

In the present invention compositions are provided having an keto-enol UV absorber and a polymeric chelator. Unlike compositions known in the related art, the polymeric chelator reduces or prevents formation of the avobenzone-iron chelate, so that the resulting insoluble complex and formula color shift are avoided. In one highly preferred embodiment of the invention, the composition also comprises one or more iron-containing adjuvants. As such, for the first time cosmetics can be created that protect the user from the full spectrum of UV-A radiation. In another highly preferred embodiment, the composition is a laundry stain-resistant sunscreen, which does not stain clothing when washed in hard (iron-containing) water.

### Keto-Enol UV Absorber

As noted above, avobenzone is one example of UV absorber that exhibits keto-enol isomerization, and it find special use in the invention. The IUPAC name of avobenzone is 1-(4-methoxyphenyl)-3-(4-tert-butylphenyl)propane-1,3-dione. Avobenzone is offered for sale under various trade names, including Escalol 517 from International Specialty Products (ISP), Parsol 1789 (DSM Nutritional Products), and Eusolex 9020 (Merck).

Other UV absorbers display the diketone moiety and are thus subject to keto-enol isomerism and chelation with iron. Additional examples of these UV absorbers are disclosed in UVA Chemical Filters: A Systematic Study, a Ph.D. dissertation by Jacqueline F. Cawthray at the University of Adelaide, Australia, 2009, which is incorporated in its entirety by reference. These diketone UV absorbers include:

acetylacetone:

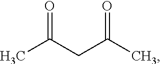

benzoylacetone:

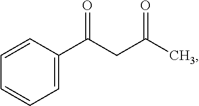 [SMILES: CC(=O)CC(=O)c1ccccc1]

dibenzoylmethane:

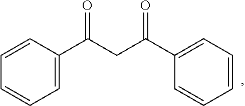 [SMILES: O=C(CC(=O)c1ccccc1)c1ccccc1]

naphthyl benzoylmethane:

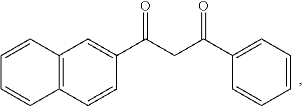 [SMILES: O=C(CC(=O)c1ccc2ccccc2c1)c1ccccc1]

and indole benzoylmethane:

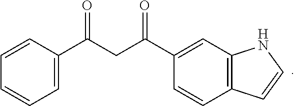 [SMILES: O=C(CC(=O)c1ccc2cc[nH]c2c1)c1ccccc1]

Additional UV absorbers having the diketone moiety can be recognized or developed by one skilled in the art. For example, the Reaxys® database on molecular structures can be queried for molecules related to those mentioned above, and suitable UV absorbers can be identified. Among these are the compounds described in the article “Synthesis of vinyl-substituted β-diketones for polymerizable metal complexes” written by Glen E. Southard and George M. Murray in J. Org. Chem., 2005, 70, 22, p. 9036-9039, which also is incorporated herein its entirety by reference. These and still other compounds like them are embraced by the present disclosure.

### Chelating Polymer

Quite to the surprise of the inventors, several families of polymers were identified that provide chelating effects. These polymers include: vinyl caprolactam/lauryl methacrylate copolymer, vinyl caprolactam/vinyl acetate copolymer, vinyl caprolactam/vinyl pyrrolidone/dimethylaminoethyl methacrylate copolymer, vinyl acetate/butyl maleate/isobornyl acrylate copolymer, and acrylic acid/vinyl pyrrolidone crosspolymer.

The functionalities of the constituent monomers in these polymers are important as they confer iron chelation. Without being bound by theory, it appears that the pseudo-cationicity of lactam moieties, like pyrrolidone and caprolactam:

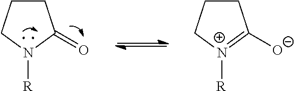

creates the necessary O⁻ anion that forms ligands with polyvalent cations like Fe²⁺ and/or Fe³⁺. By this mechanism these polymers also may provide chelation stabilization for other polyvalent cations such as Ca²⁺, Mg²⁺, and Zn²⁺. Similarly, the carboxylic acid group of mono n-butyl maleate is weakly acidic, and, like avobenzone itself (described earlier), removal of the hydroxyl proton creates a carboxylate anion that serves to form ligands with polyvalent cations like Fe²⁺ and Fe³⁺.

Given this understanding of ligand chemistry, one skilled in the art appreciates that the invention embraces a larger concept of what constitutes a polymeric chelator. Specifically, the polymeric chelator is a polymer derived from at least one monomer having at least one carboxylic acid group or one or more lactams. Highly referred are polymeric chelators derived from at least two different monomers, one having a lactam moiety and the other having a carboxylic acid moiety. Examples of these polymers and their trade names appear in Table 1.

TABLE 1
Polymeric chelators finding utility in stabilizing keto-enol UV absorbers

| generic name | trade name (supplier) |
|---|---|
| based on vinyl pyrrolidone | |
| vinyl pyrrolidone homopolymer | PVP K-15, K-30, K-60, K-90, K-120 (ISP) |
| | Kollidone ® (BASF) |
| vinyl pyrrolidone homopolymer with dimethicone | PVP/Si-10 (ISP) |
| vinyl pyrrolidone homopolymer having partially | ViviPrint ® 540 (ISP) |
| crosslinked microdomains | |
| alkylated vinyl pyrrolidone homopolymer | Ganex ® C4, C16, C20, C30 (ISP) |
| | Antaron ® C4, C16, C20, C30, V-216, V-220F, WP- |
| | 660 (all ISP) |
| vinyl pyrrolidone/vinyl acetate copolymer | E-735, I-735, W-735, W-635, S-630, E-535, |
| | I-535, E-335, I-335 (all ISP) |
| vinyl pyrrolidone/styrene copolymer | Polectron ® 430, Antara 430 (ISP) |
| vinyl pyrrolidone/acrylic acid | Acrylidone ® 1005 (ISP) |
| vinyl pyrrolidone/acrylic acid crosspolymer | Ultrathix ® P-100 (ISP) |
| vinyl pyrrolidone/dimethylaminoethyl | Copolymer 845, 937, 958 (ISP) |
| (meth)acrylate copolymer | |
| vinyl pyrrolidone/dimethylaminopropyl | Styleze ® CC-10 (ISP) |
| (meth)acrylamide | |
| vinyl pyrrolidone/acrylic acid/lauryl | Styleze ® 2000 (ISP) |
| (meth)acrylate | |
| vinyl pyrrolidone/dimethylaminoethyl | Gafquat ® 734, 755N (both ISP) |
| (meth)acrylate quaternized copolymer | |
| (polyquaternium-11) | |
| vinyl pyrrolidone/methacrylamindopropyltrimethyl | Conditioneze ® NT-20, Gafquat ® HS-100 (ISP) |
| ammonium chloride copolymer | |
| (polyquaternium-28) | |
| polyquaternium-28 with dimethicone | Gafquat ® HSi (ISP) |
| vinyl pyrrolidone/dimethylaminopropyl | Styleze ® W-10, W-20 (ISP) |
| (meth)acrylamide/methacryloylaminopropyl | |
| lauryldimonium chloride terpolymer | |
| (polyquaternium-55) | |
| vinyl pyrrolidone/dimethylaminoethyl | Copolymer 845, 937, 958 (all ISP) |
| (meth)acrylate copolymer | |
| vinyl pyrrolidone/dimethylaminopropyl | Styleze ® CC-10, ViviPrint ® 121, ViviPrint ® 131, |
| (meth)acrylamide copolymer | Setleze ® 3000 (all ISP) |
| vinyl pyrrolidone/acrylates/lauryl (meth)acrylate | Styleze ® 2000 |
| based on vinyl caprolactam | |
| vinyl caprolactam homopolymer | |
| vinyl caprolactam/vinyl acetate copolymer | |
| vinyl caprolactam/lauryl methacrylate copolymer | |
| vinyl caprolactam/vinyl pyrrolidone/ | Gaffix ® VC-713 (ISP) |
| dimethylaminoethyl (meth)acrylate terpolymer | |
| vinyl caprolactam/vinyl pyrrolidone/ | Aquaflex ® SF-40 (ISP) |
| dimethylaminopropyl (meth)acrylamide terpolymer | |
| vinyl caprolactam/dimethylaminopropyl | ViviPrint ® 200 (ISP) |
| (meth)acrylamide/hydroxyethyl (meth)acrylate | |
| polymer | |
| vinyl caprolactam/vinyl pyrrolidone/ | Advantage ® LC-E (ISP) |
| dimethylaminoethyl (meth)acrylate terpolymer and | |
| lauryl pyrrolidone | |
| vinyl caprolactam/vinyl pyrrolidone/ | Advantage ® S (ISP) |
| dimethylaminoethyl (meth)acrylate terpolymer | |
| vinyl caprolactam/vinyl pyrrolidone/ | Advantage ® HC-37 |
| (meth)acrylate terpolymer | |
| vinyl caprolactam/vinyl pyrrolidone/ | Aquastyle ® 300 |
| dimethylaminopropyl (meth)acrylamide/ | |
| methacryloylaminopropyl lauryldimonium chloride | |
| polymer | |
| vinyl methyl ether/maleic anhydride copolymer | Gantrez ® AN-119, AN-169 (ISP) |
| vinyl methyl ether/maleic di-acid copolymer | Gantrez ® S-95, S-97 (ISP) |
| vinyl methyl ether/maleic half-acid-half alkyl ester | Gantrez ® ES-225, SP-215 (ethyl ester) (ISP) |
| copolymer | Gantrez ® A-425, ES-425, ES-435 (butyl ester) |
| | (ISP) |
| | Gantrez ® ES-335 (isopropyl ester) (ISP) |
| vinyl methyl ether/maleic acid, sodium salt | Gantrez ® MS (ISP) |
| copolymer | |
| vinyl methyl ether/maleic acid decadiene | Stabileze ® (ISP) |
| crosspolymer | |
| isobutylene/dimethylaminopropyl maleimide/ | Aquaflex ® XL-30 |
| ethoxylated maleimide/maleic acid polymer | |
| (polyimide-1) | |
| methyl vinyl ether/maleic acid, monobutyl half- | EasySperse ® (ISP) |
| ester, partially neutralized with NaOH | |
| based on maleate chemistry | |
| vinyl acetate/mono n-butyl maleate/isobornyl | Advantage ® Plus (ISP) |
| (meth)acrylate copolymer | |

### Use Ratio Chelating Polymer: Keto-Enol UV Absorber

Surprisingly, the anti-chelation effect provided by the polymer is effective even at low polymer addition levels. For example, compositions prepared by the inventors successfully prevented the characteristics red-orange color change due to the complexation when 0.5% (w active/w total) of the acrylic acid/VP polymer was combined with 3.0% (w active/w total) avobenzone. Ratios as low as 0.01% polymer: 3.0% avobenzone on an active basis can be used. Higher ratios (i.e., amount of polymer) can be used and still deliver the desired UV stabilization, being limited instead by the increase in viscosity provided by the polymer. Product formats like sticks, pastes, and waxes may have ratios as high as 10% polymer:5% keto-enol UV absorbers or even higher. More preferably, ratios of 5% polymer:5% keto-enol UV absorbers are used. The most preferred compositions of the invention have around 0.5% polymer:3%-5% keto-enol UV absorber. Illustrative examples of the most preferred compositions are provided in the Examples section.

### Iron-Containing Cosmetic Adjuvants

In one preferred embodiment of the invention, the compositions also comprise one or more iron-containing cosmetic adjuvants. These compounds are generally incorporated into cosmetics for the various benefits they are known impart. The formulation chemist understands the incompatability of these adjuvants with avobenzone due to the complexes that develop. Formulation stability is facilitated, e.g., by stabilizing avobenzone in the presence of these iron-containing adjuvants.

Among these iron-containing cosmetic adjuvants the family of colorants, dyes, and pigments is preferred. These compounds are substances that serve to color the cosmetic preparation and/or to impart color to the skin or other parts of the body (e.g., hair). Examples of iron-containing colorants are: iron oxide, diiron trioxide, triiron tetraoxide, EINECS number 310-127-6 (naturally-occurring substances, mainly aluminum silicate, colored by ferric oxide), and CAS number 1302-78-9/1327-36-2/1332-58-7 (natural hydrated aluminum silicate containing calcium, magnesium or iron carbonates, ferric hydroxide, quart-sand, or mica).

Iron-containing astringents also exist and are suitable for use in the compositions disclosed herein. Astringents are compounds that tend to shrink, tighten, or constrict tissue, such as skin. Astringents having iron include iron trichloride, ferric glycerophosphate, and iron sulphate.

A number of skin conditioning agents also are employed that contain iron. Ferric citrate, the iron (2+) salt of L-aspartic acid, ferrous glucoheptonate, lactoferrin, and saccharomyces/iron ferment are a few of them.

Stabilizers, which can help prevent a compound from unwanted changes, also can contain iron, such as iron hydroxide.

Finally, the disclosed compositions also may include one or more absorbents/adsorbents, being those compounds on to which other materials (such as sebum) can absorb/adsorb One such example of an iron-based absorbed/adsorbent is calamine (zinc oxide and ferric oxide).

### Product Forms

The versatility of the invention lends the compositions to a wide variety of product formats. Yet, two general categories are generally preferred: cosmetics and sunscreens.

Because only a low amount of the chelating polymer is needed to prevent avobenzone chelation, compositions of the invention may have a low-viscosity consistency, such as a liquid, milk, lotion, or cream. Product examples of a low-viscosity compositions include sprayable sunscreens and serums, lip glosses, and lip stains. By adding more of the chelating polymer or by selecting additional thickeners, rheology modifiers, or the like, the composition may take on more body, and take for form of a thick cream, lotion, or even a gel. Numerous examples of products having this form exist, and include cosmetic foundation, concealer, blush, and bronzer. Finally, extending the polymer addition amounts, and/or adding thickeners or rheology modifiers, the composition may take the appearance of a paste, wax, stick, or even a powder. Consumers will recognize that finishing powder, lipstick, eye shadow, eyebrow pencil, and lip liner are representative examples of such product forms.

The invention's compositions may be packaged with an aerosol and be provided in the form of a mousse or a spray. To do so, it may be advantageous to utilize known propellants (e.g., hydrofluorinated compounds dichlorodifluoromethane, difluoroethane, dimethyl ether, isobutene, N-butane, propane, trichlorofluoromethane) to aide in their delivery.

In a different embodiment, compositions of this invention may be provided in the form of vaporizable fluid lotions to be applied to the skin or the hair. Pressurized devices are a suitable means for vaporizing fluid lotions, and are known to one skilled in the art. For example, they are described in U.S. Pat. Nos. 4,077,441 and 4,850,517.

The invention also contemplates compositions to serve the non-personal care arts, which are referred to broadly as performance chemical applications. Nonetheless, the product forms assume the same range in product forms, e.g., low-viscosity liquids, lotions or creams, thicker creams, lotions or gels, and pastes, waxes, sticks, and powders.

### Optional: Additional Formulation Ingredients and Adjuvants

Optional additional formulation ingredients and adjuvants may be incorporated with the UV stabilizing polymer and avobenzone UV absorber described herein. Such ingredients may be incorporated without altering the scope of the current invention, and may be included in order to produce formulated products intended for end-use applications. Some of these optional ingredients are disclosed in research disclosures IPCOM 000128968D, available at http://priorartdatabase.com/IPCOM/000128968, and IPCOM 000109682D, available at http://priorartdatabase.com/IPCOM/000109682, both of which are hereby incorporated by reference.

### UV Absorbers

In many instances it may be preferred that compositions of the invention also comprise one or more UV absorbers, particularly those that have been approved for topical use. These materials include UV-A and UV-B absorbers, such as:

p-Aminobenzoic Acid and its Derivatives:

4-aminobenzoic acid (PABA); amyl-p-dimethylaminobenzoate (padimate A), digalloyl triolate, 2,3-dihydroxypropyl 4-aminobenzoate (lisadimate, amyl dimethyl PABA, glyceryl PABA); ethyl-4-bis(hydroxypropyl)-aminobenzoate (roxadimate); ethoxylated ethyl 4-aminobenzoate (PEG-25 PABA); hexyl 2-[4-(diethylamino)-2-hydroxybenzoyl]benzoate (diethylamino hydroxy benzoyl hexylbenzoate); (5-methyl-2-propan-2-ylcyclohexyl) 2-aminobenzoate (menthyl anthranilate, meradimate);

Benzephenone Derivatives:

(2-hydroxy-4-methoxyphenyl)-(2-hydroxyphenyl)methanone (dioxybenzone); 2-hydroxy-4-methoxybenzophenone (oxybenzone, benzophenone-3); 4-hydroxy-2-methoxy-5-(oxo-phenylmethyl)benzenesulfonic acid (sulisobenzone, benzophenone-4); 2-hydroxy-4-methoxybenzophenone-5-sulphonic acid sodium salt (benzophenone-5);

Benzotriazole Derivatives:

2-(benzotriazol-2-yl)-6-[[3-(benzotriazol-2-yl)-2-hydroxy-5-(2,4,4-trimethylpentan-2-yl)phenyl]methyl]-4-(2,4,4-trimethylpentan-2-yl)phenol (bisoctrizole); 2-(2H-benzotriazol-2-yl)-4-methyl-6-[2-methyl-3-(1,3,3,3-tetramethyl-1-((trimethylsilyl)oxy)disiloxanyl)propyl)phenol (drometrizole trisiloxane); 2,2′-methylenebis-[4-(1,1,3,3-tetramethylbutyl)-6-(2H-benzotriazol-2-yl)phenol](methylene bis-benzotriazolyl tetramethylbutylphenol),

Benzimidazole Derivatives:

2-phenylbenzimidazole-5-sulfonic acid and its potassium, sodium and triethanolamine salts, (phenylbenzimidazole sulfonic acid, enzulizole); 2,2′-(1,4-phenylene)bis-1H-benzimidazole-4,6-disulfonic acid, monosodium salt (disodium phenyl dibenzimidazole tetrasulfonate);

Camphor Derivatives:

α-(2-oxoborn-3-ylidene)-toluene-4-sulphonic acid and its salts (benzylidene camphor sulfonic acid); 3,3′-(1,4-phenylenedimethylene)bis(7,7-dimethyl-2-oxobyciclo-[2.2.1]hept-1-ylmethane sulphonic acid and its salts (terephthalylidene dicamphor sulfonic acid, emcamsule); polymer of N-{(2 and 4)-[(2-oxoborn-3-ylidene)methyl]benzyl}(polyacrylamido methylbenzylidene camphor);

Cinnamates:

diethanolamine-p-methoxycinnamate (DEA methoxycinnamate);

Quinones:

lawsone with dihydroxyacetone;

Salicylates:

2-ethylhexyl salicylate (ethylhexyl salicylate); 3,3,5-trimethylcyclohexyl salicylate (homosalate, homomethyl salicylate);

Triazine Derivatives:

2,4,6-trianilino-(p-carbo-2′-ethylhexyl-1′-oxy)-1,3,5-triazine (ethylhexyl triazone); 4,4′-[[6-[[[(1,1-dimethylethyl)amino]carbonyl]phenyl]amino]-1,3,5-triazine-2,4-diyl]diimino]bis-bis(2-ethylhexyl)benzoate (diethylhexyl butamido triazone, iscotrizinol); 2,2′-[6-(4-methoxyphenyl)-1,3,5-triazine-2,4-diyl]bis[5-[(2-ethylhexyl)oxy]phenol (bis-ethylhexyloxyphenol methoxyphenyl triazine).

Especially preferred are those compositions that are formulated to provide complete protection of ultraviolet radiation. These formulations typically contain combinations of avobenzone with one or more UV-A or UV-B absorbers.

Preferred UV-A absorbers include, but not limited to: 2-aminobenzophenone, bemotrizinol, bis-benzoxazoyl phenyl ethylhexyl amino triazine, bisoctrizole, diethylaminohydroxybenzoylhexylbenzoate, diethylhexyl butamido triazone, dioxybenzone, disodium phenyl dibenzimidazole tetrasulfonate, drometrizole trisiloxane, ecamsule, ensulizole, menthyl anthranilate, meradimate, oxybenzone, sulisobenzone, and blends thereof.

Preferred embodiments of this invention that are UV-B actives include, but not limited to: aminobenzoic acid, amyl dimethyl PABA, benzophenone-9,3-benzylidene camphor sulfonic acid, bisoctrizole, camphor benzalkonium methosulfate, diethanolamine p-methoxycinnamate, diethylhexyl butamido triazone, digalloyl trioleate, drometrizole trisiloxane, ensulizole, ethyl 4-bis(hydroxypropyl)aminobenzoate, ethylhexyl p-methoxycinnamate, 2-ethylhexyl salicylate, ethylhexyl triazone, glyceryl aminobenzoate, homomethyl salicylate, lawsone with dihydroxyacetone, meradimate, methoxycinnamido propyl hydroxy sultaine, oxybenzone (benzophenone-3), 2-phenylbenzimidazole-5-sulfonic acid (and its potassium, sodium and triethanolamine salts), sulisobenzone (benzophenone-4), triethanolamine salicylate, and blends thereof.

These UV absorbers and others are identified in The Encyclopedia of Ultraviolet Filters, published by Allured Publishing Corporation in 2007. This book is hereby incorporated herein its entirety by reference.

The composition of the invention also can contain one or more additional cosmetically acceptable additives chosen from conditioning agents, protecting agents, such as, for example, hydrosoluble, antiradical agents, antioxidants, vitamins and pro-vitamins, fixing agents, insect repellants, oxidizing agents, reducing agents, dyes, cleansing agents, anionic, cationic, nonionic and amphoteric surfactants, thickeners, perfumes, pearlizing agents, stabilizers, pH adjusters, filters, preservatives, hydroxy acids, cationic and nonionic polyether associative polyurethanes, polymers other than the cationic polymer described herein, vegetable oils, mineral oils, synthetic oils, polyols such as glycols and glycerol, silicones, aliphatic alcohols, colorants, bleaching agents, highlighting agents and sequestrants. These additives are present in the composition according to the invention in proportions that may range from 0 to 20% by weight in relation to the total weight of the composition. The precise amount of each additive may be easily determined by an expert in the field according to its nature and its function.

### Conditioning Agents

Any known conditioning agent is useful in the personal care compositions of this invention. Conditioning agents function to improve the cosmetic properties of the hair, particularly softness, thickening, untangling, feel, and static electricity and may be in liquid, semi-solid, or solid form such as oils, waxes, or gums. Similarly, any known skin altering agent is useful in the compositions of this invention. Preferred conditioning agents include cationic polymers, cationic surfactants and cationic silicones.

Conditioning agents may be chosen from synthesis oils, mineral oils, vegetable oils, fluorinated or perfluorinated oils, natural or synthetic waxes, silicones, cationic polymers, proteins and hydrolyzed proteins, ceramide type compounds, cationic surfactants, fatty amines, fatty acids and their derivatives, as well as mixtures of these different compounds.

The synthesis oils include polyolefins, e.g., poly-α-olefins such as polybutenes, polyisobutenes and polydecenes. The polyolefins can be hydrogenated.

The mineral oils suitable for use in the compositions of the invention include hexadecane and oil of paraffin.

A list of suitable animal and vegetable oils comprises sunflower, corn, soy, avocado, jojoba, squash, raisin seed, sesame seed, walnut oils, fish oils, glycerol tricaprocaprylate, Purcellin oil or liquid jojoba, and blends thereof.

Suitable natural or synthetic oils include eucalyptus, lavender, vetiver, litsea cubeba, lemon, sandalwood, rosemary, chamomile, savory, nutmeg, cinnamon, hyssop, caraway, orange, geranium, cade, and bergamot.

Suitable natural and synthetic waxes include carnauba wax, candelila wax, alfa wax, paraffin wax, ozokerite wax, vegetable waxes such as olive wax, rice wax, hydrogenated jojoba wax, absolute flower waxes such as black currant flower wax, animal waxes such as bees wax, modified bees wax (cerabellina), marine waxes and polyolefin waxes such as polyethylene wax, and blends thereof.

The cationic polymers that may be used as a conditioning agent according to the invention are those known to improve the cosmetic properties of hair treated by detergent compositions. The expression “cationic polymer” as used herein, indicates any polymer containing cationic groups and/or ionizable groups in cationic groups. The cationic polymers used generally have a molecular weight the average number of which falls between about 500 and 5,000,000 and preferably between 1000 and 3,000,000.

The preferred cationic polymers are chosen from among those containing units including primary, secondary, tertiary, and/or quaternary amine groups that may either form part of the main polymer chain or a side chain.

Useful cationic polymers include known polyamine, polyaminoamide, and quaternary polyammonium types of polymers, such as:

(1) homopolymers and copolymers derived from acrylic or methacrylic esters or amides. The copolymers can contain one or more units derived from acrylamides, methacrylamides, diacetone acrylamides, acrylamides and methacrylamides, acrylic or methacrylic acids or their esters, vinyllactams such as vinyl pyrrolidone or vinyl caprolactam, and vinyl esters. Specific examples include: copolymers of acrylamide and dimethyl amino ethyl methacrylate quaternized with dimethyl sulfate or with an alkyl halide; copolymers of acrylamide and methacryloyl oxyethyl trimethyl ammonium chloride; the copolymer of acrylamide and methacryloyl oxyethyl trimethyl ammonium methosulfate; copolymers of vinyl pyrrolidone/dialkylaminoalkyl acrylate or methacrylate, optionally quaternized, such as the products sold under the name Gafquat® by International Specialty Products; the dimethyl amino ethyl methacrylate/vinyl caprolactam/vinyl pyrrolidone terpolymers, such as the product sold under the name Gaffix® VC 713 by International Specialty Products; the vinyl pyrrolidone/methacrylamidopropyl dimethylamine copolymer, marketed under the name Styleze® CC 10 by International Specialty Products; and the vinyl pyrrolidone/quaternized dimethyl amino propyl methacrylamide copolymers such as the product sold under the name Gafquat® HS 100 by International Specialty Products (Wayne, N.J.).

(2) derivatives of cellulose ethers containing quaternary ammonium groups, such as hydroxy ethyl cellulose quaternary ammonium that has reacted with an epoxide substituted by a trimethyl ammonium group.

(3) derivatives of cationic cellulose such as cellulose copolymers or derivatives of cellulose grafted with a hydrosoluble quaternary ammonium monomer, as described in U.S. Pat. No. 4,131,576, such as the hydroxy alkyl cellulose, and the hydroxymethyl-, hydroxyethyl- or hydroxypropyl-cellulose grafted with a salt of methacryloyl ethyl trimethyl ammonium, methacrylamidopropyl trimethyl ammonium, or dimethyl diallyl ammonium.

(4) cationic polysaccharides such as described in U.S. Pat. Nos. 3,589,578 and 4,031,307, guar gums containing cationic trialkyl ammonium groups and guar gums modified by a salt, e.g., chloride of 2,3-epoxy propyl trimethyl ammonium.

(5) polymers composed of piperazinyl units and alkylene or hydroxy alkylene divalent radicals with straight or branched chains, possibly interrupted by atoms of oxygen, sulfur, nitrogen, or by aromatic or heterocyclic cycles, as well as the products of the oxidation and/or quaternization of such polymers.

(6) water-soluble polyamino amides prepared by polycondensation of an acid compound with a polyamine These polyamino amides may be reticulated.

(7) derivatives of polyamino amides resulting from the condensation of polyalcoylene polyamines with polycarboxylic acids followed by alcoylation by bi-functional agents.

(8) polymers obtained by reaction of a polyalkylene polyamine containing two primary amine groups and at least one secondary amine group with a dioxycarboxylic acid chosen from among diglycolic acid and saturated dicarboxylic aliphatic acids having 3 to 8 atoms of carbon. Such polymers are described in U.S. Pat. Nos. 3,227,615 and 2,961,347.

(9) the cyclopolymers of alkyl dialyl amine or dialkyl diallyl ammonium such as the homopolymer of dimethyl diallyl ammonium chloride and copolymers of diallyl dimethyl ammonium chloride and acrylamide.

(10) quaternary diammonium polymers such as hexadimethrine chloride.

(11) quaternary polyammonium polymers, including, for example, Mirapol® A 15, Mirapol® AD1, Mirapol® AZ1, and Mirapol® 175 products sold by Miranol.

(12) the quaternary polymers of vinyl pyrrolidone and vinyl imidazole such as the products sold under the names Luviquat® FC 905, FC 550, and FC 370 by BASF Corp. (Ludwigshafen, DE).

(13) quaternary polyamines

(14) reticulated polymers known in the art.

Other cationic polymers that may be used within the context of the invention are cationic proteins or hydrolyzed cationic proteins, polyalkyleneimines such as polyethyleneimines, polymers containing vinyl pyridine or vinyl pyridinium units, condensates of polyamines and epichlorhydrins, quaternary polyurethanes, and derivatives of chitin.

Preferred cationic polymers are derivatives of quaternary cellulose ethers, the homopolymers and copolymers of dimethyl diallyl ammonium chloride, quaternary polymers of vinyl pyrrolidone and vinyl imidazole, and mixtures thereof.

The conditioning agent can be any silicone known by those skilled in the art to be useful as a conditioning agent. The silicones suitable for use according to the invention include polyorganosiloxanes that are insoluble in the composition. The silicones may be present in the form of oils, waxes, resins, or gums. They may be volatile or non-volatile. The silicones can be selected from polyalkyl siloxanes, polyaryl siloxanes, polyalkyl aryl siloxanes, silicone gums and resins, and polyorgano siloxanes modified by organofunctional groups, and mixtures thereof.

Suitable polyalkyl siloxanes include polydimethyl siloxanes with terminal trimethyl silyl groups or terminal dimethyl silanol groups (dimethiconol) and polyalkyl (C₁-C₂₀) siloxanes.

Suitable polyalkyl aryl siloxanes include polydimethyl methyl phenyl siloxanes and polydimethyl diphenyl siloxanes, linear or branched.

The silicone gums suitable for use herein include polydiorganosiloxanes preferably having a number-average molecular weight between 200,000 and 1,000,000, used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxane.

Suitable silicone resins include silicones with a dimethyl/trimethyl siloxane structure and resins of the trimethyl siloxysilicate type.

The organo-modified silicones suitable for use in the invention include silicones such as those previously defined and containing one or more organofunctional groups attached by means of a hydrocarbon radical and grafted siliconated polymers. Particularly preferred are amino functional silicones.

The silicones may be used in the form of emulsions, nano-emulsions, or micro-emulsions.

The conditioning agent can be a protein or hydrolyzed cationic or non-cationic protein. Examples of these compounds include hydrolyzed collagens having triethyl ammonium groups, hydrolyzed collagens having trimethyl ammonium and trimethyl stearyl ammonium chloride groups, hydrolyzed animal proteins having trimethyl benzyl ammonium groups (benzyltrimonium hydrolyzed animal protein), hydrolyzed proteins having groups of quaternary ammonium on the polypeptide chain, including at least one C₁-C₁₈ alkyl.

Hydrolyzed proteins include Croquat L, in which the quaternary ammonium groups include a C₁₋₂ alkyl group, Croquat M, in which the quaternary ammonium groups include C₁₀-C₁₈ alkyl groups, Croquat S in which the quaternary ammonium groups include a C₁₋₈ alkyl group and Crotein Q in which the quaternary ammonium groups include at least one C₁-C₁₈ alkyl group. These products are sold by Croda.

The conditioning agent can comprise quaternized vegetable proteins such as wheat, corn, or soy proteins such as cocodimonium hydrolyzed wheat protein, laurdimonium hydrolyzed wheat protein and steardimonium hydrolyzed wheat protein.

2-N-stearoyl amino-octadecane-1,3-diol, 2-N-behenoyl amino-octadecane-1,3-diol, 2-N-[2-hydroxy-palmitoyl]-amino-octadecane-1,3-diol, 2-N-stearoyl amino-octadecane-1,3,4-triol, N-stearoyl phytosphingosine, 2-N-palmitoyl amino-hexadecane-1,3-diol, bis-(N-hydroxy ethyl N-cetyl) malonamide, N-(2-hydroxy ethyl)-N-(3-cetoxyl-2-hydroxy propyl) amide of cetylic acid, N-docosanoyl N-methyl-D-glucamine and mixtures of such compounds.

The conditioning agent can be a cationic surfactant such as a salt of a primary, secondary, or tertiary fatty amine, optionally polyoxyalkylenated, a quaternary ammonium salt, a derivative of imadazoline, or an amine oxide. Suitable examples include mono-, di-, or tri-alkyl quaternary ammonium compounds with a counterion such as a chloride, methosulfate, tosylate, etc. including, but not limited to, cetrimonium chloride, dicetyldimonium chloride, behentrimonium methosulfate, and the like. The presence of a quaternary ammonium compound in conjunction with the polymer described above reduces static and enhances combing of hair in the dry state. The polymer also enhances the deposition of the quaternary ammonium compound onto the hair substrate thus enhancing the conditioning effect of hair.

The conditioning agent can be any fatty amine known to be useful as a conditioning agent; e.g. dodecyl, cetyl or stearyl amines, such as stearamidopropyl dimethylamine

The conditioning agent can be a fatty acid or derivatives thereof known to be useful as conditioning agents. Suitable fatty acids include myristic acid, palmitic acid, stearic acid, behenic acid, oleic acid, linoleic acid, and isostearic acid. The derivatives of fatty acids include carboxylic ester acids including mono-, di-, tri- and tetra-carboxylic acids.

The conditioning agent can be a fluorinated or perfluorinated oil. The fluoridated oils may also be fluorocarbons such as fluoramines, e.g., perfluorotributylamine, fluoridated hydrocarbons, such as perfluorodecahydronaphthalene, fluoroesters, and fluoroethers.

Of course, mixtures of two or more conditioning agents can be used.

The conditioning agent or agents can be present in an amount of 0.001% to 20%, preferably from 0.01% to 10%, and even more preferably from 0.1% to 3% by weight based on the total weight of the final composition.

### Emollients, Humectants and Moisturizers

Because many of the invention's compositions find utility on the skin, emollients, humectants, and moisturizers find special usefulness. Compounds that soften and smooth the skin are generally referred to as emollients. Humectants are understand by one skilled in the art to be compounds that hold and/or retain moisture, while moisturizers increase skin's water content and help keep it soft and smooth. Often, an additive will serve more than one of these roles. Representative examples of emollient, humectants, and moisturizers can be found in the International Cosmetic Ingredient Dictionary and Handbook, Ninth Edication, edited by Pepe, Wenninger, and McEwen, 2002, the infobase of the Personal Care Products Council, and the Inventory and Common Nomenclature of Ingredients Employed in Cosmetic Products (dated 9 Feb. 2006), all of which are hereby incorporated herein their entirety by reference.

### Anti-Oxidants and the Like

The composition of the invention can contain one or more protecting agents to prevent or limit the degrading effects of natural physical and/or chemical assaults on the keratinous materials.

The antioxidants or antiradical agents can be selected from phenols such as BHA (tert-butyl-4-hydroxy anisole), BHT (2,6-di-tert-butyl-p-cresol), TBHQ (tert-butyl hydroquinone), polyphenols such as proanthocyanodic oligomers, flavonoids, hindered amines such as tetra amino piperidine, erythorbic acid, polyamines such as spermine, cysteine, glutathione, superoxide dismutase, and lactoferrin.

The vitamins can be selected from ascorbic acid (vitamin C), vitamin E, vitamin E acetate, vitamin E phosphate, B vitamins such as B3 and B5, vitamin PP, vitamin A, and derivatives thereof. The provitamins can be selected from panthenol and retinol.

The protecting agent can be present in an amount 0.001% to 20% by weight, preferably from 0.01% to 10% by weight, and more preferably 0.1 to 5% by weight of the total weight of the final composition.

### Surfactants

In addition, the compositions according to the invention advantageously include at least one surfactant, which can be present in an amount of 0.1% and 60% preferably 1% and 40%, and more preferably 5% and 30% by weight based on the total weight of the composition. The surfactant may be chosen from among anionic, amphoteric, or non-ionic surfactants, or mixtures of them known to be useful in personal care compositions.

### Thickeners

Additional thickeners or viscosity increasing agents may be included in the composition of the invention, such as: Acetamide MEA; acrylamide/ethalkonium chloride acrylate copolymer; acrylamide/ethyltrimonium chloride acrylate/ethalkonium chloride acrylate copolymer; acrylamides copolymer; acrylamide/sodium acrylate copolymer; acrylamide/sodium acryloyldimethyltaurate copolymer; acrylates/acetoacetoxyethyl methacrylate copolymer; acrylates/beheneth-25 methacrylate copolymer; acrylates/C₁₀-C₃₀ alkyl acrylate crosspolymer; acrylates/ceteth-20 itaconate copolymer; acrylates/ceteth-20 methacrylate copolymer; acrylates/laureth-25 methacrylate copolymer; acrylates/palmeth-25 acrylate copolymer; acrylates/palmeth-25 itaconate copolymer; acrylates/steareth-50 acrylate copolymer; acrylates/steareth-20 itaconate copolymer; acrylates/steareth-20 methacrylate copolymer; acrylates/stearyl methacrylate copolymer; acrylates/vinyl isodecanoate crosspolymer; acrylic acid/acrylonitrogens copolymer; adipic acid/methyl DEA crosspolymer; agar; agarose; alcaligenes polysaccharides; algin; alginic acid; almondamide DEA; almondamidopropyl betaine; aluminum/magnesium hydroxide stearate; ammonium acrylates/acrylonitrogens copolymer; ammonium acrylates copolymer; ammonium acryloyldimethyltaurate/vinyl formamide copolymer; ammonium acryloyldimethyltaurate/VP copolymer; ammonium alginate; ammonium chloride; ammonium polyacryloyldimethyl taurate; ammonium sulfate; amylopectin; apricotamide DEA; apricotamidopropyl betaine; arachidyl alcohol; arachidyl glycol; arachis hypogaea (peanut) flour; ascorbyl methylsilanol pectinate; astragalus gummifer gum; attapulgite; avena sativa (oat) kernel flour; avocadamide DEA; avocadamidopropyl betaine; azelamide MEA; babassuamide DEA; babassuamide MEA; babassuamidopropyl betaine; behenamide DEA; behenamide MEA; behenamidopropyl betaine; behenyl betaine; bentonite; butoxy chitosan; caesalpinia spinosa gum; calcium alginate; calcium carboxymethyl cellulose; calcium carrageenan; calcium chloride; calcium potassium carbomer; calcium starch octenylsuccinate; C20-40 alkyl stearate; canolamidopropyl betaine; capramide DEA; capryl/capramidopropyl betaine; carbomer; carboxybutyl chitosan; carboxymethyl cellulose acetate butyrate; carboxymethyl chitin; carboxymethyl chitosan; carboxymethyl dextran; carboxymethyl hydroxyethylcellulose; carboxymethyl hydroxypropyl guar; carnitine; cellulose acetate propionate carboxylate; cellulose gum; ceratonia siliqua gum; cetearyl alcohol; cetyl alcohol; cetyl babassuate; cetyl betaine; cetyl glycol; cetyl hydroxyethylcellulose; chimyl alcohol; cholesterol/HDI/pullulan copolymer; cholesteryl hexyl dicarbamate pullulan; citrus aurantium dulcis (orange) peel extract; cocamide DEA; cocamide MEA; cocamide MIPA; cocamidoethyl betaine; cocamidopropyl betaine; cocamidopropyl hydroxysultaine; coco-betaine; coco-hydroxysultaine; coconut alcohol; coco/oleamidopropyl betaine; coco-Sultaine; cocoyl sarcosinamide DEA; cornamide/cocamide DEA; cornamide DEA; croscarmellose; crosslinked bacillus/glucose/sodium glutamate ferment; cyamopsis tetragonoloba (guar) gum; decyl alcohol; decyl betaine; dehydroxanthan gum; dextrin; dibenzylidene sorbitol; diethanolaminooleamide DEA; diglycol/CHDM/is ophthalates/SIP copolymer; dihydroabietyl behenate; dihydrogenated tallow benzylmonium hectorite; dihydroxyaluminum aminoacetate; dimethicone/PEG-10 crosspolymer; dimethicone/PEG-crosspolymer; dimethicone propyl PG-betaine; dimethylacrylamide/acrylic acid/polystyrene ethyl methacrylate copolymer; dimethylacrylamide/s odium acryloyldimethyltaurate crosspolymer; disteareth-100 IPDI; DMAPA acrylates/acrylic acid/acrylonitrogens copolymer; erucamidopropyl hydroxysultaine; ethylene/sodium acrylate copolymer; gelatin; gellan gum; glyceryl alginate; glycine soja (soybean) flour; guar hydroxypropyltrimonium chloride; hectorite; hyaluronic acid; hydrated silica; hydrogenated potato starch; hydrogenated tallow; hydrogenated tallowamide DEA; hydrogenated tallow betaine; hydroxybutyl methylcellulose; hydroxyethyl acrylate/sodium acryloyldimethyl taurate copolymer; hydroxyethylcellulose; hydroxyethyl chitosan; hydroxyethyl ethylcellulose; hydroxyethyl stearamide-MIPA; hydroxylauryl/hydroxymyristyl betaine; hydroxypropylcellulose; hydroxypropyl chitosan; hydroxypropyl ethylenediamine carbomer; hydroxypropyl guar; hydroxypropyl methylcellulose; hydroxypropyl methylcellulose stearoxy ether; hydroxypropyl starch; hydroxypropyl starch phosphate; hydroxypropyl xanthan gum; hydroxystearamide MEA; isobutylene/sodium maleate copolymer; isostearamide DEA; isostearamide MEA; isostearamide mIPA; isostearamidopropyl betaine; lactamide MEA; lanolinamide DEA; lauramide DEA; lauramide MEA; lauramide MIPA; lauramide/myristamide DEA; lauramidopropyl betaine; lauramidopropyl hydroxysultaine; laurimino bispropanediol; lauryl alcohol; lauryl betaine; lauryl hydroxysultaine; lauryl/myristyl glycol hydroxypropyl ether; lauryl sultaine; lecithinamide DEA; linoleamide DEA; linoleamide MEA; linoleamide MIPA; lithium magnesium silicate; lithium magnesium sodium silicate; macrocystis pyrifera (kelp); magnesium alginate; magnesium/aluminum/hydroxide/carbonate; magnesium aluminum silicate; magnesium silicate; magnesium trisilicate; methoxy PEG-22/dodecyl glycol copolymer; methylcellulose; methyl ethylcellulose; methyl hydroxyethylcellulose; microcrystalline cellulose; milkamidopropyl betaine; minkamide DEA; minkamidopropyl betaine; MIPA-myristate; montmorillonite; Moroccan lava clay; myristamide DEA; myristamide MEA; myristamide MIPA; myristamidopropyl betaine; myristamidopropyl hydroxysultaine; myristyl alcohol; myristyl betaine; natto gum; nonoxynyl hydroxyethylcellulose; oatamide MEA; oatamidopropyl betaine; octacosanyl glycol isostearate; octadecene/MA copolymer; oleamide DEA; oleamide MEA; oleamide MIPA; oleamidopropyl betaine; oleamidopropyl hydroxysultaine; oleyl betaine; olivamide DEA; olivamidopropyl betaine; oliveamide MEA; palmamide DEA; palmamide MEA; palmamide MIPA; palmamidopropyl betaine; palmitamide DEA; palmitamide MEA; palmitamidopropyl betaine; palm kernel alcohol; palm kernelamide DEA; palm kernelamide MEA; palm kernelamide MIPA; palm kernelamidopropyl betaine; peanutamide MEA; peanutamide MIPA; pectin; PEG-800; PEG-crosspolymer; PEG-150/decyl alcohol/SMDI copolymer; PEG-175 diisostearate; PEG-190 distearate; PEG-15 glyceryl tristearate; PEG-140 glyceryl tristearate; PEG-240/HDI copolymer bis-decyltetradeceth-20 ether; PEG-100/IPDI copolymer; PEG-180/laureth-50/TMMG copolymer; PEG-10/lauryl dimethicone crosspolymer; PEG-15/lauryl dimethicone crosspolymer; PEG-2M; PEG-5M; PEG-7M; PEG-9M; PEG-14M; PEG-20M; PEG-23M; PEG-25M; PEG-45M; PEG-65M; PEG-90M; PEG-115M; PEG-160M; PEG-180M; PEG-120 methyl glucose trioleate; PEG-180/octoxyno1-40/TMMG copolymer; PEG-150 pentaerythrityl tetrastearate; PEG-4 rapeseedamide; PEG-150/stearyl alcohol/SMDI copolymer; phaseolus angularis seed powder; polianthes tuberosa extract; polyacrylate-3; polyacrylic acid; polycyclopentadiene; polyether-1; polyethylene/isopropyl maleate/MA copolyol; polyglyceryl-3 disiloxane dimethicone; polyglyceryl-3 polydimethylsiloxyethyl dimethicone; polymethacrylic acid; polyquaternium-52; polyvinyl alcohol; potassium alginate; potassium aluminum polyacrylate; potassium carbomer; potassium carrageenan; potassium chloride; potassium palmate; potassium polyacrylate; potassium sulfate; potato starch modified; PPG-2 cocamide; PPG-1 hydroxyethyl caprylamide; PPG-2 hydroxyethyl cocamide; PPG-2 hydroxyethyl coco/isostearamide; PPG-3 hydroxyethyl soyamide; PPG-14 laureth-60 hexyl dicarbamate; PPG-14 laureth-60 isophoryl dicarbamate; PPG-14 palmeth-60 hexyl dicarbamate; propylene glycol alginate; PVP/decene copolymer; PVP montmorillonite; pyrus cydonia seed; pyrus malus (apple) fiber; rhizobian gum; ricebranamide DEA; ricinoleamide DEA; ricinoleamide MEA; ricinoleamide MIPA; ricinoleamidopropyl betaine; ricinoleic acid/adipic acid/AEEA copolymer; rosa multiflora flower wax; sclerotium gum; sesamide DEA; sesamidopropyl betaine; sodium acrylate/acryloyldimethyl taurate copolymer; sodium acrylates/acrolein copolymer; sodium acrylates/acrylonitrogens copolymer; sodium acrylates copolymer; sodium acrylates crosspolymer; sodium acrylate/sodium acrylamidomethylpropane sulfonate copolymer; sodium acrylates/vinyl isodecanoate crosspolymer; sodium acrylate/vinyl alcohol copolymer; sodium carbomer; sodium carboxymethyl chitin; sodium carboxymethyl dextran; sodium carboxymethyl beta-glucan; sodium carboxymethyl starch; sodium carrageenan; sodium cellulose sulfate; sodium chloride; sodium cyclodextrin sulfate; sodium hydroxypropyl starch phosphate; sodium isooctylene/MA copolymer; sodium magnesium fluorosilicate; sodium oleate; sodium palmitate; sodium palm kernelate; sodium polyacrylate; sodium polyacrylate starch; sodium polyacryloyldimethyl taurate; sodium polygamma-glutamate; sodium polymethacrylate; sodium polystyrene sulfonate; sodium silicoaluminate; sodium starch octenylsuccinate; sodium stearate; sodium stearoxy PG-hydroxyethylcellulose sulfonate; sodium styrene/acrylates copolymer; sodium sulfate; sodium tallowate; sodium tauride acrylates/acrylic acid/acrylonitrogens copolymer; sodium tocopheryl phosphate; solanum tuberosum (potato) starch; soyamide DEA; soyamidopropyl betaine; starch/acrylates/acrylamide copolymer; starch hydroxypropyltrimonium chloride; stearamide AMP; stearamide DEA; stearamide DEA-distearate; stearamide DIBA-stearate; stearamide MEA; stearamide MEA-stearate; stearamide MIPA; stearamidopropyl betaine; steareth-60 cetyl ether; steareth-100/PEG-136/HDI copolymer; stearyl alcohol; stearyl betaine; sterculia urens gum; synthetic fluorphlogopite; tallamide DEA; tallow alcohol; tallowamide DEA; tallowamide MEA; tallowamidopropyl betaine; tallowamidopropyl hydroxysultaine; tallowamine oxide; tallow betaine; tallow dihydroxyethyl betaine; tamarindus indica seed gum; tapioca starch; TEA-alginate; TEA-carbomer; TEA-hydrochloride; trideceth-2 carboxamide MEA; tridecyl alcohol; triethylene glycol dibenzoate; trimethyl pentanol hydroxyethyl ether; triticum vulgare (wheat) germ powder; triticum vulgare (wheat) kernel flour; triticum vulgare (wheat) starch; tromethamine acrylates/acrylonitrogens copolymer; tromethamine magnesium aluminum silicate; undecyl alcohol; undecylenamide DEA; undecylenamide MEA; undecylenamidopropyl betaine; welan gum; wheat germamide DEA; wheat germamidopropyl betaine; xanthan gum; yeast beta-glucan; yeast polysaccharides and zea mays (corn) starch.

Preferred thickeners or viscosity increasing agents include carbomer, aculyn and Stabileze®, e.g. crosslinked acrylic acid, crosslinked poly(methylvinyl ether/maleic anhydride) copolymer, acrylamides, carboxymethyl cellulose and the like.

Aspects of the present application will be described in more detail by reference to the following non-limiting examples.

### EXAMPLES

### Example 1

To illustrate the chelation properties of several polymers, aqueous preparations were made containing 100 ppm of various polymers and from 2 ppm to 25 ppm iron (designated D). For each preparation, the amount of free iron (D_{f}) and bound iron were measured, and then the percentage of free iron not bound was calculated as D_{f}/D.

As shown in Table 2, six polymers—VCAP/LMA copolymer, VCAP/VA copolymer, vinyl caprolactam/VP/dimethylaminoethyl methacrylate copolymer, VA/butyl maleate/isobornyl acrylate copolymer, acrylic acid/VP crosspolymer—were discovered to be chelating polymers, as they significantly reduced the amount of free iron. Preferred chelating polymers achieve a value of D_{f}/D of 50% or less. More preferred chelating polymers reduce this ratio to 25% or less, while the most preferred chelating polymers result in only 10% or less free iron. The most exemplary chelating polymers reduce the amount of free iron to 5% or less.

TABLE 2
Free and bound iron data of Example 1

| | concentration (ppm) | | | |
|---|---|---|---|---|
| | total iron | | bound iron | |
| chelator | (D) | free iron | (D_{b}) | $\frac{{D}_{f}}{D}$ |
| avobenzone | 2 | 0.20 | 1.80 | 10% |
| | 4 | 0.17 | 3.83 | 4% |
| | 5 | 0.18 | 4.83 | 3% |
| | 15 | 0.21 | 14.79 | 1% |
| | 25 | 8.43 | 16.57 | 34% |
| vinyl caprolactam / | 2 | 0.18 | 1.82 | 9% |
| lauryl methacrylate | 4 | 0.17 | 3.83 | 4% |
| copolymer | 5 | 0.17 | 4.83 | 3% |
| | 15 | 0.24 | 14.76 | 2% |
| | 25 | 1.14 | 23.86 | 5% |
| vinyl caprolactam / | 2 | 0.17 | 1.83 | 8% |
| vinyl acetate | 4 | 0.17 | 3.83 | 4% |
| copolymer | 5 | 0.17 | 4.83 | 3% |
| | 15 | 0.23 | 14.77 | 2% |
| | 25 | 0.99 | 24.01 | 4% |
| vinyl caprolactam / VP / | 2 | 1.51 | 0.49 | 75% |
| dimethylaminoethyl | 4 | 2.03 | 1.97 | 51% |
| methacrylate | 5 | 0.15 | 4.85 | 3% |
| copolymer | 15 | 0.32 | 14.68 | 2% |
| (Advantage ® S) | 25 | 0.83 | 24.17 | 3% |
| VA / mono n-butyl | 2 | 1.84 | 0.16 | 48% |
| maleate / isobornyl | 4 | 0.88 | 3.12 | 46% |
| acrylate copolymer | 5 | 0.15 | 4.85 | 38% |
| (Advantage ® Plus) | 15 | 0.17 | 14.83 | 25% |
| | 25 | 0.82 | 24.18 | 26% |
| acrylic acid / VP | 5 | 0.18 | | 4% |
| crosspolymer | 10 | 0.24 | | 2% |
| (Ultrathix ® P-100) | 25 | 2.93 | | 12% |

### Example 2

A UV-absorbing foundation was prepared having the ingredients shown in Table 3. First, the water, butylene glycol, and disodium EDTA were mixed until clear, and then the triethanolamine was added and mixed until uniform. Then, the acrylic acid/VP crosspolymer (Ultrathix® P-100) was sprinkled into the mixture and blended for one hour until it was completely hydrated. Meanwhile, the ingredients of phase B were weighed, mixed, and pulverized for about 10 minutes. In a separate container, the ingredients of phase C were added, mixed, and heated to 75° C.-80° C. After reaching temperature, the mixer for phase C was replaced by a homogenizer, and phase B was added, and homogenized at 75° C.-80° C. Similarly, phase A was heated to 75° C.-80° C., and then phase B+C was added to phase A, and homogenized for about 10 minutes. Afterward, the batch was cooled to 55° C., and then phase D was added with homogenization. Then, sweep agitation was used and the batch cooled to 35° C. Phase E then was added with mixing until it reached room temperature (about 25° C.).

The pH of the batch was 6.35. The viscosity was measured at 5 rpm using a Brookfield LVT-TC viscometer and found to be 100,000 cP.

TABLE 3
The foundation formula of Example 2

| | | | | addition |
|---|---|---|---|---|
| | | | | level |
| phase | INCI name | trade name | supplier | (% w/w) |
| A | water | deionized water | | 37.59 |
| | butylene glycol | butylene glycol | | 4.00 |
| | disodium EDTA | disodium EDTA | | 0.10 |
| | triethanolamine | triethanolamine | | 0.38 |
| | acrylic Acid/VP | Ultrathix ® P-100 | ISP | 0.50 |
| | crosspolymer | | | |
| B | silica | SB700 silica beads | US | 1.00 |
| | | | Cosmetics | |
| | | | Corp. | |
| | | BTD-401 ITT treated TiO₂ | Kobo | 6.78 |
| | | BYO-12 ITT treated yellow iron oxide | Kobo | 0.65 |
| | | BRO-12 ITT treated red iron oxide | Kobo | 0.25 |
| | | BBO-12 ITT treated black iron oxide | Kobo | 0.14 |
| | | O-13 ITT treated sericite | Kobo | 1.56 |
| C | glyceryl stearate (and) | Cerasynt ® 945 | ISP | 3.00 |
| | laureth-23 | | | |
| | ceteareth-20 | Emulgin ® B2 | Cognis | 1.00 |
| | decyl oleate | Ceraphyl ® 140 | ISP | 1.50 |
| | isocetyl stearate | Ceraphyl ® 494 | ISP | 0.75 |
| | octocrylene | Escalol ® 597 | ISP | 8.00 |
| | benzophenone-3 | Escalol ® 567 | ISP | 5.00 |
| | ethylhexyl salycilate | Escalol ® 587 | ISP | 5.00 |
| | avobenzone | Escalol ® 517 | ISP | 3.00 |
| | dimethicone | Si Tec DM350 | ISP | 1.00 |
| | phenyl trimethicone | Si Tec PTM 20 | ISP | 12.00 |
| | trimethylsiloxysilicate | Si Tec TMS 803 | ISP | 0.30 |
| D | cyclopentasiloxane | Si Tec CM040 | ISP | 5.00 |
| E | caprylyl glycol, | Optiphen ® | ISP | 1.50 |
| | phenoxyethanol | | | |
| | | | total | 100.00 |

### Example 3

The method of Example 2 was followed to create a second foundation of the invention (Table 4). This formulation contained no ethylhexyl salycilate, and 2% less octocrylene and 2% less benzophenone-3 than the formula of Example 1.

The pH of the batch was 6.08. The viscosity was measured at 5 rpm using a Brookfield LVT-TC viscometer and found to be 34,000 cP.

TABLE 4
The foundation formula of Example 3

| | | | | addition |
|---|---|---|---|---|
| | | | | level |
| phase | INCI name | trade name | supplier | (% w/w) |
| A | water | | | 57.14 |
| | butylene glycol | | | 4.00 |
| | disodium EDTA | | | 0.10 |
| | triethanolamine | | | 0.38 |
| | acrylic acid/VP | Ultrathix ® P-100 | ISP | 0.50 |
| | crosspolymer | | | |
| B | silica | SB700 silica beads | US | 1.00 |
| | | | Cosmetics | |
| | | | Corp. | |
| | | BTD-401 ITT treated TiO₂ | Kobo | 6.78 |
| | | BYO-12 ITT treated yellow iron oxide | Kobo | 0.65 |
| | | BRO-12 ITT treated red iron oxide | Kobo | 0.25 |
| | | BBO-12 ITT treated black iron oxide | Kobo | 0.14 |
| | | O-13 ITT treated sericite | Kobo | 1.56 |
| C | glyceryl stearate (and) | Cerasynt ® 945 | ISP | 3.00 |
| | laureth-23 | | | |
| | ceteareth-20 | Emulgin ® B2 | Cognis | 1.00 |
| | decyl oleate | Ceraphyl ® 140 | ISP | 1.50 |
| | isocetyl stearate | Ceraphyl ® 494 | ISP | 0.75 |
| | octocrylene | Escalol ® 597 | ISP | 6.00 |
| | benzophenone-3 | Escalol ® 567 | ISP | 3.00 |
| | avobenzone | Escalol ® 517 | ISP | 3.00 |
| | phenyl trimethicone | Si Tec PTM 20 | ISP | 4.00 |
| | trimethylsiloxysilicate | Si Tec TMS 803 | ISP | 0.50 |
| D | cyclopentasiloxane | Si Tec CM040 | ISP | 4.00 |
| E | phenoxyethanol, | Liquapar ® PE | ISP | 0.75 |
| | isopropylparaben, | | | |
| | isobutylparaben, n- | | | |
| | butylparaben | | | |
| | | | total | 100.00 |

### Examples 4 and 5

Two sunscreens were prepared having the formulas summarized in Table 5. The sunscreens contained avobenzone in combination with VA/butyl maleate/isobornyl acrylate copolymer; other UV absorbers were included. The sunscreens did not contain water, but instead had ethanol. Also, iron-containing adjuvants were not incorporated into the formulas. A control sunscreen was made having the same ingredients but without any chelating polymer (the balance added as ethanol).

The two sunscreens of the invention and the control were applied to cotton and cotton/poly fabric swatches and allowed to dry overnight. Then, the swatches were laundered in 100° F. water with a commercial laundry detergent using a tergotometer, and then rinsed in fresh water. After air drying, the swatches were compared to the original, untested fabric swatches. Those swatches stained with sunscreen formulas #4 and #5 displayed considerably less staining after washing than the control.

TABLE 5
The sunscreen formulas of Examples 4 and 5

| | addition level (w/w) | |
|---|---|---|
| ingredients | #4 | #5 |
| avobenzone (Escalol ® 517) | 3.0% | 3.0% |
| oxybenzone (Escalol ® 567) | 6.0% | 6.0% |
| homosalate (Eusolex ® HMS) | 15.0% | 15.0% |
| octisalate (Escalol ® 587) | 5.0% | 5.0% |
| octocrylene (Escalol ® 597) | 10.0% | 10.0% |
| ethanol | 60.0% | 59.0% |
| vinyl caprolactam/vinyl pyrrolidone/ | 1.0% | |
| dimethylaminoethyl (meth)acrylate terpolymer | | |
| (Advantage ® S) | | |
| VA/butyl maleate/isobornyl acrylate copolymer | | 2.0% |
| (Advantage ® Plus) | | |
| total | 100.0% | 100.0% |

What we claim is:

1. A UV-absorbing composition comprising: (A) a keto-enol UV absorber and (B) a chelating polymer.

2. The composition according to claim 1 wherein said keto-enol UV absorber is selected from the group consisting of: avobenzone, acetylacetone, benzoylacetone, dibenzoylmethane, naphthyl benzoylmethane, indole benzoylmethane, and combinations thereof.

3. The composition of claim 2 wherein said keto-enol UV absorber is avobenzone.

4. The composition according to claim 1 having from 0.1% to 10% (w active/w total) of said keto-enol UV absorber.

5. The composition according to claim 1 wherein said chelating polymer is derived from at least one monomer having lactam functionality or carboxylic acid functionality.

6. The composition according to claim 5 wherein said lactam functionality is selected from the group consisting of: pyrrolidone, caprolactam, and combinations thereof.

7. The composition according to claim 5 wherein said chelating polymer is selected from the group consisting of: vinyl caprolactam/lauryl methacrylate copolymer, vinyl caprolactam/vinyl acetate copolymer, vinyl caprolactam/VP/dimethylaminoethyl methacrylate copolymer, VA/mono n-butyl maleate/isobornyl acrylate copolymer, acrylic acid/VP crosspolymer, and combinations thereof.

8. The composition according to claim 1 having from 0.01% to 10.0% (w active/w total) of said chelating polymer.

9. The composition according to claim 1 that is a: sunscreen, a sprayable sunscreen, a moisturizer, skin cream, ointment, liniment, anti-aging cream, lip moisturizer, lip gloss, hair spray, hair finishing spray, mousse, hair styling gel, hair styling cream, or hair styling wax.

10. The composition according to claim 1 that further comprises one or more iron-containing compounds.

11. The composition according to claim 10 wherein said iron-containing compound is selected from the group consisting of: colorants, dyes, and pigment; astringents; skin conditioning agents; stabilizers, absorbents and adsorbents; and combinations thereof.

12. The composition according to claim 11 wherein said iron-containing compound is selected from the group consisting of: iron oxide, diiron trioxide, triiron tetraoxide, naturally-occurring substances, mainly aluminum silicate, colored by ferric oxide (EINECS number 310-127-6, and CAS number 1302-78-9/1327-36-2/1332-58-7 (natural hydrated aluminum silicate containing calcium, magnesium or iron carbonates, ferric hydroxide, quart-sand, or mica), iron trichloride, ferric glycerophosphate, iron sulphate, ferric citrate, the iron (2+) salt of L-aspartic acid, ferrous glucoheptonate, lactoferrin, saccharomyces/iron ferment, iron hydroxide, calamine, and combinations thereof.

13. The composition according to claim 10 having from 0.001% to 10% (w active/w total) of said iron-containing compound.

14. The composition according to claim 13 that is selected from the group consisting of: serums, lip glosses, lip stains, cosmetic foundations, concealers, blushes, bronzers, finishing powders, lipsticks, eye shadows, eye brow pencils, and lip liners.

15. The composition of claim 14 that additionally comprises at least one ingredient selected from the group consisting of: actives, adhesives, anti-oxidants, binders, biocides, colorants, defoamers, dyes, emollients, fragrances, humectants, insect repellants, UV absorbers, lubricants, moisturizers, oils, pigments, preservatives, propellants, surfactants, thickeners, water, waxes, and blends thereof.

16. The composition according to claim 15 wherein said additional UV absorber is selected from the group consisting of: octocrylene, cinoxate, 3-benzylidene camphor, 4-methylbenzylidene camphor, lisadimate, benzophenone-1, benzophenone-3; benzophenone-4; benzophenone-5; benzophenone-6, benzophenone-8, benzophenone-9, bisdisulizole disodium, bemotrizinol, bisoctrizole, camphor benzalkonium methosulfate, DEA methoxycinnamate, diethylaminohydroxybenzoylhexyl benzoate, iscotrizinol, digalloyl trioleate, diisopropyl methylcinnamate, dimethoxyphenyl-[1-(3,4)]-4,4-dimethyl 1,3-pentanedione, drometrizole, drometrizole trisiloxane, roxadimate, ethylhexyl-p-methoxycinnamate, ethylhexyl triazone, ferulic acid, glyceryl ethylhexanoate dimethoxycinnamate, homosalate, isoamyl-p-methoxycinnamate, lawsone with dihydroxyacetone, meradimate, padimate 0, octyl salicylate, PABA, PEG-25 PABA, padimate A, ensulizole, polyacrylamido methylbenzylidene camphor, polysilicone-15, encamsule, trolamine salicylate, titanium dioxide, zinc oxide, and combinations thereof.
